(19) United States
(12) Patent Application Publication
(10) Pub. No.: US 2025/0172502 A1
(43) Pub. Date: May 29, 2025

(54) MEASUREMENT CHIP, MEASURING DEVICE AND MEASURING METHOD

(71) Applicant: Furuno Electric Co., Ltd., Nishinomiya-City (JP)

(72) Inventors: Keiji TADA, Kobe (JP); Takeshi KAWAJIRI, Kawanishi (JP); Syoichiro KAJI, Osaka (JP)

(73) Assignee: Furuno Electric Co., Ltd., Nishinomiya-City (JP)

(21) Appl. No.: 19/039,844

(22) Filed: Jan. 29, 2025

Related U.S. Application Data
(63) Continuation of application No. PCT/JP2023/030418, filed on Aug. 24, 2023.

(30) Foreign Application Priority Data
Nov. 25, 2022 (JP) 2022-188273

(51) Int. Cl. G01N 21/77; G01N 33/543; G02B 6/12; G02B 6/124

(52) U.S. Cl. CPC G01N 21/7743; G01N 33/54373; G02B 6/124; G02B 2006/12107

(57) ABSTRACT

Embodiments of present disclosure provide an optical waveguide type measurement chip with further improved measurement stability, a measurement device and a measurement method. The measurement chip comprises a propagation layer configured to allow light to propagate, an introductory part configured to have a first diffraction grating for introducing the light into the propagation layer, an outgoing part configured to have a second diffraction grating for deriving the light from the propagation layer, and a ligand modification surface configured to be a surface of the propagation layer and capable of modifying a ligand that reacts with an analyte be detected, wherein a period of a plurality of grating patterns formed in at least one of: the first diffraction grating, or the second diffraction grating, is different from each other between two or more regions.

### CROSS-REFERENCE TO RELATED APPLICATION(S)

This application is a continuation application of PCT International Application No. PCT/JP2023/030418, which was filed on Aug. 24, 2023, and which claims priority to Japanese Patent Application No. JP2022-188273 filed on Nov. 25, 2022, the entire disclosures of each of which are herein incorporated by reference for all purposes.

### TECHNICAL FIELD

The present disclosure generally relates to an optical waveguide type measurement chip, more specifically, the present disclosure relates to measurement chips, measuring devices and measuring methods for measuring for measuring a change in a pattern of light.

### BACKGROUND

Biosensors are analytical devices that combine a biological component with a physicochemical detector to detect and measure presence of specific biological or chemical substances. These devices are designed to convert biological responses into measurable signals, enabling quantification of various analytes in a sample. Biosensors find applications in diverse fields, including medical diagnostics, environmental monitoring, food safety, and more.

Various kinds of biosensors have been developed, for example, to analyze interactions between biomolecules. Example of different types of biosensors may include, but is not limited to, optical waveguides-based biosensors, surface plasmon resonance-based biosensors, and Mach-Zehnder interference-based biosensors.

In an example, an optical waveguide type biosensor (also referred to as, an optical waveguide type measurement chip) may use an optical waveguide in order to detect a substance (such as, an analyte) to be measured. In this regard, a reactant (such as, a ligand) reacts with the analyte is formed on the surface of a propagation layer where the light propagates.

However, the conventional measurement chip of the optical waveguide type requires further performance improvement in terms of measurement stability.

### SUMMARY

As described in detail below, there is a need for further performance improvements in measurement stability of conventional optical waveguide type biosensors or measurement chips.

It is an object of the present disclosure to provide a measurement chip of the optical waveguide type, a measuring device, and a measuring method with improved measurement stability.

To achieve the above object, the present invention includes, for example, the following aspects.

In one aspect, the present disclosure provides a measurement chip. The measurement chip comprises a propagation layer configured to allow light to propagate, an introductory part configured to have a first diffraction grating for introducing the light into the propagation layer, an outgoing part configured to have a second diffraction grating for outgoing of the light from the propagation layer, and a ligand modification surface configured to be a surface of the propagation layer. The ligand modification surface is capable of modifying a ligand that reacts with an analyte be detected. Moreover, a period of a plurality of grating patterns formed in at least one of: the first diffraction grating, or the second diffraction grating, is different from each other between two or more regions.

According to an embodiment, the period of the plurality of grating patterns formed in the first diffraction grating is different from the period of the plurality of grating patterns formed in the second diffraction grating between two or more regions, such that the two or more regions lie along a propagation direction of the light.

According to an embodiment, the first diffraction grating is configured to have the period of the plurality of grating patterns, such that the period increases or decreases for each of the two or more regions along the propagation direction of the light.

According to an embodiment, the first diffraction grating is configured to have the period of the plurality of grating patterns, such that the period increases or decreases for each of the two or more regions along the propagation direction of light while maintaining a constant duty ratio.

According to an embodiment, the period of the plurality of grating patterns formed in the second diffraction grating is constant along the propagation direction of the light.

According to an embodiment, an average value of the periods of the plurality of grating patterns of the first diffraction grating is different from the an average value of the periods of the plurality of grating patterns of the second diffraction grating.

According to an embodiment, the measurement chip is further configured to include a plurality of sets of each of: the introductory part, the propagation layer, the outgoing part and the ligand modification surface. The ligand is modified on one of the plurality of sets of the ligand modification surface. In an example, periods of the plurality of grating patterns formed on the second diffraction grating are different among the plurality of sets.

According to an embodiment, the first diffraction grating is further configured to have a planar shape of the plurality of grating patterns with reducing coupling efficiency at both ends in a direction perpendicular to the propagation direction of the light.

According to an embodiment, a phase distribution of the light changes due to a change in a refractive index around the propagation layer caused by a reaction between the analyte and the ligand.

In another aspect, a measuring device is provided. The measurement device includes a measurement chip. The measurement chip comprises a propagation layer configured to allow light to propagate, an introductory part configured to have a first diffraction grating for introducing the light into the propagation layer, an outgoing part configured to have a second diffraction grating for outgoing of the light from the propagation layer, and a ligand modification surface configured to be a surface of the propagation layer. The ligand modification surface is capable of modifying a ligand that reacts with an analyte be detected. Moreover, a period of a plurality of grating patterns formed in the first diffraction grating is different from a period of a plurality of grating patterns formed in the second diffraction grating between two or more regions. The measurement device further include a light source configured to guide the light to the introductory part of the measuring chip, a photodetector configured to receive the light derived from the outgoing part of the measurement chip, and a processing circuitry configured to analyze a change in a pattern of the light received by the photodetector.

According to an embodiment, the processing circuitry is further configured to analyze a change in a traveling direction of the light.

According to an embodiment, a collimator lens is configured to be positioned

between the light source and the introductory part, and collimate light emitted from the light source is configured to illuminate a plurality of introductory parts.

According to an embodiment, the collimator lens is further configured to be positioned in an optical system that is out of collimate condition.

In yet another aspect, a measurement method is provided. The measurement method may be performed using a measurement chip. The measurement method comprises introducing light into a propagation layer, causing the light to gent totally reflected in the propagation layer. The propagation layer has a ligand layer on a surface of the propagation layer that reacts with an analyte to be detected. The measurement method further comprises causing outgoing of the light from the propagation layer.

According to an embodiment, the measuring method further comprises analyzing changes in a pattern of the light outgone from the propagation layer.

### EFFECT(S) OF THE INVENTION

Embodiments to the present disclosure provide an optical waveguide type measurement chip, a measurement device and a measurement method with improved measurement reproducibility and measurement reliability.

### BRIEF DESCRIPTION OF DRAWINGS

The embodiments of the present disclosure is illustrated by way of example and not by way of limitation in the figures of the accompanying drawings, in which the like reference numerals indicate like elements and in which:

FIGS. 1(A)-1(C) show illustrations of a schematic structure of a measurement chip, according to an embodiment of the present disclosure;

FIGS. 2(A) and 2(B) show illustrations of a schematic diagram to explain a

principle of measurement by the measurement chip, according to an example embodiment of the present disclosure;

FIG. 3 illustrates a schematic diagram to show a configuration of a measurement device, according to an example embodiment of the present disclosure;

FIG. 4 illustrates a flowchart of a measurement method, according to an example embodiment of the present disclosure;

FIGS. 5(A)-5(C) illustrate diagrams showing a schematic structure of the measurement device and the measurement chip, according to another embodiment of the present disclosure;

FIGS. 6(A) and 6(B) illustrate diagrams showing a schematic structure of the measurement device and the measurement chip, according to yet another embodiment of the present disclosure;

FIGS. 7(A) and 7(B) illustrate diagrams showing the schematic structure of the measurement device and the measurement chip, according to another embodiment of the present disclosure;

FIG. 8 illustrates a diagram showing the schematic structure of the measurement device, according to yet another embodiment of the present disclosure;

FIGS. 9(A) and 9(B) illustrate planar views showing a variation of diffraction grating provided at the measurement chip, according to an embodiment of the present disclosure;

FIG. 10 is a graph showing allowable range of incident angle, θ, of light, according to an embodiment of the present disclosure;

FIGS. 11(A) and 11(B) illustrate diagrams showing the schematic structure of the measurement chip indicating deviation of peak angle of the incident angle of the light, according to an embodiment of the present disclosure;

FIGS. 12(A)-12(C) illustrates diagrams showing a schematic structure of a conventional optical waveguide type measurement chip, according to an embodiment; and

FIG. 13 illustrates a diagram showing the schematic structure of a measurement device when multiple measurement objects are measured in parallel using the conventional optical waveguide type measurement chip, according to an embodiment.

### DETAILED DESCRIPTION OF THE EMBODIMENTS

Embodiments of the invention will be described in detail below with reference to the accompanying drawings. It is to be noted that in the following descriptions and drawings, the same symbols refer to the same or similar components, and thus duplicate descriptions of the same or similar components will be omitted.

In the following description, for purposes of explanation, numerous specific details are set forth in order to provide a thorough understanding of the present disclosure. It will be apparent, however, to one skilled in the art that the present disclosure may be practiced without these specific details. In other instances, systems and methods are shown in block diagram form only in order to avoid obscuring the present disclosure.

Some embodiments of the present disclosure will now be described more fully hereinafter with reference to the accompanying drawings, in which some, but not all, embodiments of the disclosure are shown. Indeed, various embodiments of the disclosure may be embodied in many different forms and should not be construed as limited to the embodiments set forth herein; rather, these embodiments are provided so that this disclosure will satisfy applicable legal requirements. Like reference numerals refer to like elements throughout. Also, reference in this specification to “one embodiment” or “an embodiment” means that a particular feature, structure, or characteristic described in connection with the embodiment is included in at least one embodiment of the present disclosure. The appearance of the phrase “in one embodiment” in various places in the specification are not necessarily all referring to the same embodiment, nor are separate or alternative embodiments mutually exclusive of other embodiments. Further, the terms “a” and “an” herein do not denote a limitation of quantity, but rather denote the presence of at least one of the referenced items. Moreover, various features are described which may be exhibited by some embodiments and not by others. Similarly, various requirements are described which may be requirements for some embodiments but not for other embodiments.

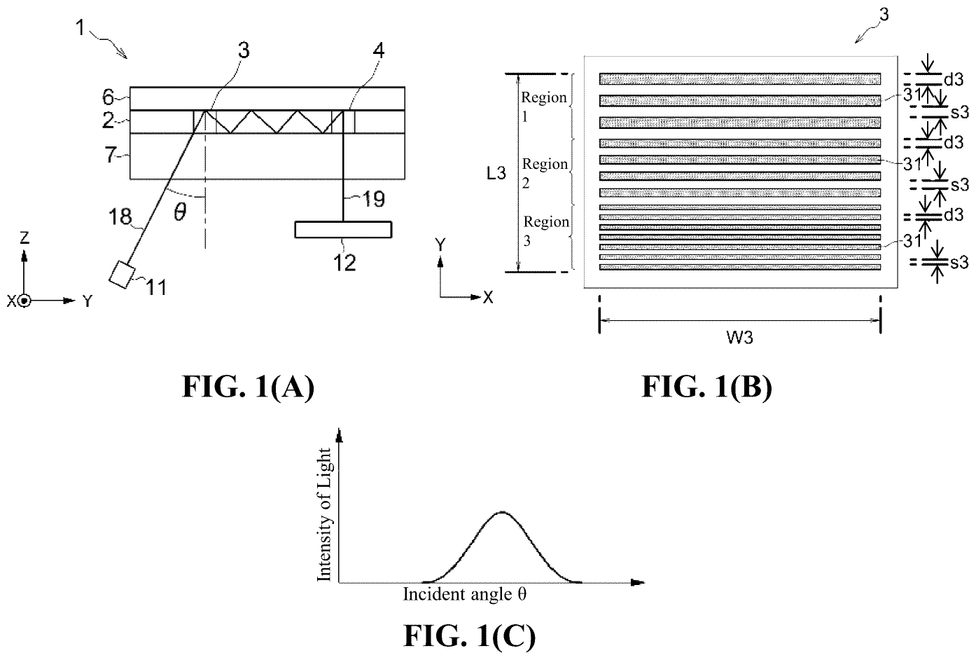

The embodiments are described herein for illustrative purposes and are subject to many variations. It is understood that various omissions and substitutions of equivalents are contemplated as circumstances may suggest or render expedient but are intended to cover the application or implementation without departing from the spirit or the scope of the present disclosure. Further, it is to be understood that the phraseology and terminology employed herein are for the purpose of the description and should not be regarded as limiting. Any heading utilized within this description is for convenience only and has no legal or limiting effect. Turning now to FIG. 1-FIG. 13, a brief description concerning the various components of the present disclosure will now be briefly discussed. Reference will be made to the figures showing various embodiments of a measurement chip comprising a ligand layer on a surface of a propagation layer that reacts with an analyte to be detected.

In the figures, a top surface direction (thickness direction) of a measurement chip is defined as Z-axis direction, a light propagation direction in the measurement chip is defined as a Y-axis direction, and a direction perpendicular to the light propagation direction is defined as an X-axis direction. References to the term “surface” indicates either a top or a bottom surface, and references to the term “both surfaces” indicates both the top and the bottom surfaces.

### First Embodiment

### Measurement Chip

FIGS. 1(A)-1(C) show illustrations of a schematic structure of a measurement chip 1, according to an embodiment. FIG. 1(A) illustrates a side view of the measurement chip 1. FIG. 1(B) illustrates a top view of a diffraction grating provided at an introductory part 3 of the measurement chip 1. FIG. 1(C) illustrates a view showing a relationship between an intensity of light derived from the introductory part 3 and an incident angle of the light.

An outline of the measurement chip 1 according to the first embodiment will be described with reference to FIGS. 1(A)-1(C).

Referring to FIG. 1(A), the measurement chip 1 includes a propagation layer 2 where the light propagates. The measurement chip 1 includes an introductory part 3 that introduces the light into the propagation layer 2, and an outgoing part 4 that derives the light out from the propagation layer 2. Moreover, the measurement chip 1 includes a ligand-modified surface on a surface of the propagation layer 2. The ligand-modified surface may modify a ligand (such as, antibodies) that reacts with an analyte (such as, an antigen) in an object to be measured (such as, a specimen). Further, the measurement chip 1 includes a ligand layer 6. The ligand layer 6 may be formed when the ligand is modified on the ligand-modified surface of the surface of the propagation layer 2 when or before the measurement chip 1 is used for measurement.

In operation, when the measurement chip 1 is used for measurement, light 18 is emitted from a light source 11 and introduced into an inside of the propagation layer 2 from an underside of the measurement chip 1 through the introductory part 3. The light 18 may undergo total reflection inside the propagation layer 2, and light 19 is then derived out of the propagation layer 2 from the underside of the measurement chip 1 through the outgoing part 4 and is received by a photodetector 12.

Referring to FIG. 1(B), a first diffraction grating of the introductory part 3 is shown. The introductory part 3 uses the first diffraction grating having a plurality of grating patterns 31. Further, periods of the grating patterns 31 may be present in the Y-axis direction of the multiple grating patterns 31. Pursuant to present example, the periods of the grating patterns 31 are different from each other between two or more regions.

In an example, a period of a grating pattern refers to a sum of a width of the grating pattern and a spacing of the grating pattern. It may be noted, the Y-axis direction is the direction in which the light 18 propagates in the propagation layer 2. In the present example, the periods of the grating patterns 31 may vary from region to region. An effect of using such first diffraction grating is described below.

For example, when the light 18 strikes the first diffraction grating of the introductory part 3, the light 18 diffracts at a diffraction angle. The diffraction angle is determined based on a wavelength of the light 18, a surrounding refractive index, an incident angle, θ, of the light 18 and a period of the diffraction gratings. In other words, when the light 18 is irradiated to the first diffraction grating, multiple diffracted light with slightly different diffraction angles may be generated in each region. For example, if the light 18 is irradiated to the first diffraction grating of the introductory part 3 at the incident angle, θ, and the incident light 18 is coupled to a propagation mode with high efficiency in region 2 then coupling efficiency to the propagation mode is not high in regions 1 and 3 adjacent to the region 2. This happens because, in a waveguide with a sufficiently thin propagation layer 2 (e.g., about 50 nanometers (nm)) as in the present embodiment, propagating light may exist only in a very narrow range of angles centered on a specific angle dependent on by the wavelength of the light 18 and the surrounding refractive index (i.e., the refractive index of the surrounding materials).

Further, when the incident angle, θ, deviates to an angle represented as, θ+Δθ, (e.g., Δθ may be 0.6 degrees), then the coupling efficiency to the propagation mode in the region 2 decreases greatly, but the coupling efficiency in the region 3 increases, so the overall coupling efficiency across the regions of the propagation layer 2 does not change significantly. As a result, as shown in FIG. 1(C), an allowable range of the incident angle, θ, of light 18 incident on the introductory part 3 is expanded (approximately by 4.0 degrees, for example).

For example, if a thickness of the propagation layer 2 is varied in a range of ±5 nm, approximately, then an optimum value of the incident angle, θ, may have a variation of about +1.2°. Furthermore, for example, when a temperature of a semiconductor laser used as the light source 11 is varied between 5° C. and 40° C., the wavelength of the light 18 emitted from the light source 11 varies in a range of about, for example, ±5.25 nm. To this end, when a variation in the thickness of the propagation layer 2 and the variation in the temperature of the semiconductor laser are combined, the optimum incident angle, θ, has a variation of about, for example, +2.0°. However, since the allowable range of the incident angle, θ, is, for example, 4.0 degrees in the measurement chip 1, the measurement of the analyte may be performed without any problem even with these variations.

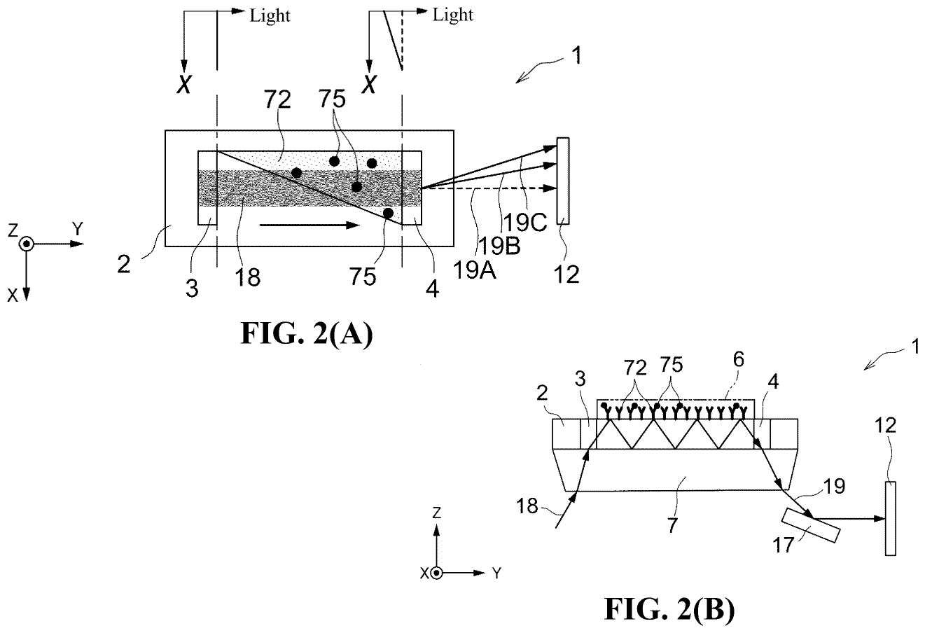

FIGS. 2(A) and 2(B) show illustrations of a schematic diagram for explaining a principle of measurement by the measurement chip 1, according to the present embodiment. FIG. 2(A) shows a top view of the measurement chip 1. FIG. 2(B) shows a side view of the measurement chip 1.

When used for measurement or before the use, the measurement chip 1 includes a ligand 72 that may be modified on the surface of the propagation layer 2 (referred to as, the ligand-modified surface) to form a ligand layer 6 on the surface of the propagation layer 2. A flow path or a flow channel is provided on an upper surface of the measurement chip 1 on which the ligand layer 6 is formed. In the flow path, a solution of the object to be measured (referred to as an object, hereinafter) flows. The object contains an analyte 75.

In an example, the light 18 is introduced into the propagation layer 2 through the introductory part 3. The light 18 propagates in the Y-axis direction in the propagation layer 2, and is derived out from the propagation layer 2 through the outgoing part 4. For example, the light 19 that is derived out from the propagation layer 2 through the outgoing part 4 is received by the photodetector 12. In an example, the photodetector 12 may have its optical axis adjusted appropriately by a mirror 17 or the like. The mirror 17 may have an arbitrary configuration.

While propagating in the Y-axis direction in the propagation layer 2, the light 18 is affected by refractive index change due to a reaction (or binding) between the analyte 75 in the object flowing in the flow path and the ligand 72 in the ligand layer 6. In the measurement chip 1, the ligand layer 6 is formed on the surface of the propagation layer 2 such that a length of the ligand layer 6 extends along the light propagation direction (Y-axis direction). Moreover, the length of the ligand layer 6 increases or decreases along a direction (X-axis direction) perpendicular to the propagation direction. Thus, when the refractive index changes on the surface of the propagation layer 2 where the ligand layer 6 is formed due to the reaction between the analyte 75 and the ligand 72, a phase distribution (in the X-axis direction) of the light propagating in the Y-axis direction also changes. To this end, when the phase distribution of the light changes, a traveling direction of the light changes.

Therefore, the light 18 propagating in the Y-axis direction in the propagation layer 2 may follow an optical path 19A, for example, when neither ligand 72 nor analyte 75 is present on the surface of the propagation layer 2. For example, when only ligand 72 is present, an optical path 19B may be followed. It may be noted, the optical path 19B may be deviated from the optical path 19 A. For example, when both ligand 72 and analyte 75 are present, an optical path 19 C may be followed, such that the optical path 19C is deviated from the optical path 19B.

Thus, the traveling direction of the light 19 derived from the outgoing part 4 changes due to the influence of the refractive index change. Such refractive index change may occur due to the reaction of ligand 72 and analyte 75. Therefore, by performing analytical processing to analyze the change in the traveling direction of the light 19, it becomes possible to estimate a presence or an absence of the analyte 75, concentration of analyte 75, and kinetic parameters (Kinetics) of the analyte 75 in the object or the solution of the object to be measured.

Details of each part of the measurement chip 1 are described below, for example, with reference to FIGS. 1(A)-1(C).

The propagation layer 2 is flat. Light 18 is introduced into the propagation layer 2 from the introductory part 3. The light 18 may be totally reflected on the upper and lower surfaces of the propagation layer 2, and is derived out from the outgoing part 4. In an embodiment, the propagation layer 2 may be made of an evaporated film (for example, a material having a refractive index of about 2.07, or depending on the wavelength of light 18). For example, the propagation layer 2 may be made of metal oxides, such as titanium oxide (TiO₂) and tantalum oxide (Ta₂O₅). Further, for manufacturing of the propagation layer 2, dielectrics such as acrylic resin, glass, polyvinyl alcohol, polyvinyl chloride, silicone resin or polystyrene may also be used in addition to the evaporated film. For example, a thickness ‘d’ of the propagation layer 2 in the Z-axis direction may be in a range of, for example, 50 nm to 100 nm. Moreover, a length of the propagation layer 2 in the Y-axis direction may be, for example, 4 mm. in addition, a length of the propagation layer 2 in the X-axis direction may be in a range of, for example, 270 micrometers (μm) to 600 μm. When a dielectric, such as acrylic resin, glass, polyvinyl alcohol, polyvinyl chloride, silicone resin, or polystyrene is used for manufacturing of the propagation layer 2, the propagation layer 2 itself may function as a base material, and a base material 7 of the measurement chip 1 may be omitted.

The introductory part 3 and the outgoing part 4 are provided in the propagation layer 2. According to present embodiment, a diffraction grating is used for the introductory part 3 and the outgoing part 4. In an example, there may be phase-type and amplitude-type diffraction gratings, and phase-type diffraction gratings may be made by, for example, a Nano-imprint method. Also, amplitude-type diffraction gratings may be made by, for example, electron beam drawing and deposition of light-shielding materials such as chromium.

Pursuant to present example, a phase-type diffraction grating may be provided at the introductory part 3 and the outgoing part 4, but this should not be construed as a limitation. In other embodiments, the same effect of present disclosure may be obtained by using an amplitude-type diffraction grating.

In an example, the first diffraction grating of the introductory part 3 may have a length L3 in the Y-axis direction. For example, the length L3 may be approximately 100 microns. Moreover, the first diffraction grating of the introductory part 3 may have a length W3 in the X-axis direction. For example, the length W3 may be approximately 270 microns.

Referring back to FIG. 1(B), the multiple grating patterns 31 of the first diffraction grating provided in the introductory part 3 are different from each other between two or more regions in terms of the periods. A period may be indicated by a sum of a width d3 and a spacing s3) in the Y-axis direction of the multiple grating patterns 31. A period of a grating pattern may be expressed by a sum of a width of an adjacent grating pattern and a spacing of the grating pattern. In one embodiment, the first diffraction grating is phase-type and the grating patterns 31 may be realized using grooves. In another embodiment, the first diffraction grating is amplitude-type and the grating patterns 31 may be realized using shading sections.

In an example, the periods of the grating patterns 31 refer to periods of grooves in the case of phase-type diffraction grating and periods of shading sections in the case of amplitude-type diffraction grating. It may be noted, periods of the grating patterns 31 may vary from region to region, and even within each region. For example, the periods of the multiple grating patterns 31 provided in the introductory part 3 are different from each other between two or more regions along the direction in which light propagates, i.e., the Y-axis direction. Moreover, the first diffraction grating provided in the introductory part 3 has the periods of the multiple grating patterns 31. Such periods may increase or decrease for each region along the direction in which light propagates, i.e., the Y-axis direction.

In an example, the periods of the first diffraction grating provided in the introductory part 3 increases or decreases for each region along the direction in which light propagates while maintaining a duty ratio. The duty ratio represents a ratio of length. The duty ratio may be expressed as d3/(d3+s3) where d3 is the width of the grating patterns 31 and s3 is the spacing of the grating patterns 31. In the example, for a grating pattern from the multiple grating patterns 31, if the width d3 of an adjacent grating pattern and the spacing s3 of the grating pattern have same dimensions, then the duty ratio may be, for example, 0.5.

In an example, in case of a phase-type grating, a cross-sectional shape of the grating patterns 31 of the first diffraction grating, or one periodic portion of the grating patterns 31 (including an adjacent d3 and s3) may be, for example, saw-shaped, rectangular, triangular, trapezoidal and semicircular. In another example, in case of an amplitude-type grating, shading portions of the diffraction gratings 31 may correspond to a film formed of a metallic material, such as chromium to a certain thickness (e.g., 50 nm or more). In particular, a phase-type grating in which a cross-sectional shape of a periodic portion of a grating pattern is saw-shaped is called a blazed grating. It may be noted that such blazed grating may improve diffraction efficiency. When a cross-sectional shape of a grating pattern is rectangular, a length of the width d3 of the grating pattern may be defined, for example, at an approximate center of a depth of the grating pattern. For example, the depth of the grating pattern may be about, for example, half of the width d3 of the grating pattern.

In an example, the ligand layer 6 is formed by modification to the ligand-modified surface of the surface of the propagation layer 2. It may be noted, the ligand 72 may refer to a substance that reacts or binds specifically to an analyte 75. The analyte 75 is a substance to be detected or measured in the object. In an example, a refractive index of the ligand layer 6 is about 1.33.

In an embodiment, the ligand layer 6 is formed in a planar shape, for example, right-angled triangles in planar view. For example, a length of the ligand layer 6 may extend in the light propagation direction, i.e., the Y-axis direction. Moreover, the length of the ligand layer 6 may increase or decrease along a direction, i.e., the X-axis direction, that is perpendicular to the light propagation direction. In another embodiment, the ligand layer 6 is formed in a stripe shape on the surface of the propagation layer 2.

Continuing further, ligand content in the light propagation direction or the Y-axis direction varies monotonically along the X-axis direction perpendicular to the light propagation direction, in the ligand layer 6 of a portion located on the surface of the propagation layer 2. Thus, when the refractive index changes due to the reaction (or binding) between the analyte 75 and the ligand 72 on the surface of the propagation layer 2 where the ligand layer 6 is formed, the phase distribution of the light propagating in the Y-axis direction changes in the X-axis direction.

To this end, the measurement chip 1 functions as a measurement chip for estimating the presence or absence of the analyte 75 and/or estimating the concentration or kinetic parameters of the analyte 75. In an example, the ligand content may be calculated by multiplying ligand content density per unit length in the light propagation direction with the length of the ligand layer 6 along the light propagation direction.

In an example, the base material 7 may be a transparent substrate. The base material 7 may have an arbitrary configuration and is provided on the lower surface of the propagation layer 2. For example, the base material may be made of glass (such as, having a refractive index of about 1.47˜1.48). In another embodiment, the measurement chip 1 may further include an intermediate layer, such as a fluororesin, between the lower surface of the propagation layer 2 and the base material or the substrate 7.

### Measurement Apparatus

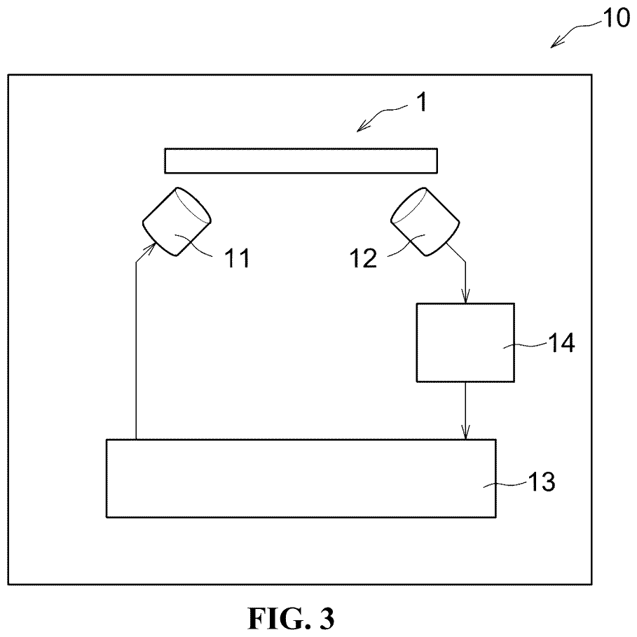

FIG. 3 illustrates a schematic diagram showing a configuration of a measurement device 10, according to the first embodiment.

The measurement device 10 may include a light source 11 that guides the light 18 to the introductory part 3 of the measurement chip 1, a photodetector 12 that receives the light 19 derived from the introductory part 4 of the measurement chip 1, and a control unit 13 (which is also referred to as a processing circuitry) that analyzes a change in a pattern of the light 19 received by the photodetector 12. The pattern of the light 19 received by the photodetector 12 changes when the object to be measured contacts the measurement chip 1 and the ligand 72 and the analyte 75 reacts. In an example, the measurement device 10 further includes a measuring unit 14 that acquires intensity information of the light received by each light receiving element of the photodetector 12.

The control unit 13 and the measuring unit 14 may be configured in hardware using, for example, a dedicated integrated circuit (IC), or implemented in software using an information processing device such as a general-purpose computer, a smartphone, or a tablet terminal.

The measurement chip 1 is placed at a predetermined location in the measuring device 10. Light emitted from the light source 11 is introduced into the propagation layer 2 from the lower surface of the measurement chip 1 through the introductory part 3. The light totally reflected inside the propagation layer 2 is derived from the lower surface of the measurement chip 1 through the outgoing part 4 and received by the photodetector 12.

In an example, the light source 11 emits visible light of a wavelength of, for example, 650 nm. For example, a wavelength range of the light emitted by the light source 11 may be in a range of, for example, 450 nm to 2000 nm. In an example, the light emitted by the light source 11 is a Gaussian beam. A Gaussian beam is suitable for detecting changes in the light pattern (or intensity distribution) because a rough shape of the light pattern does not change as the light propagates. In an example, the light emitted by the light source is a continuous wave. It may be noted, the Gaussian beam may be two-dimensional, such as in the X-axis direction and the Z-axis direction, however, this should not be construed as a limitations. In other example, the Gaussian beam may be one-dimensional, such as in the X-axis direction. For such a light source 11 for emitting a Gaussian beam, for example, a semiconductor laser device may be used.

The photodetector 12 receives the light 19 derived from the outgoing part 4. In an example, the photodetector 12 is composed of light receiving elements arranged in one or two dimensions. Various image sensors such as a charge-coupled device (CCD) image sensor or a complementary metal oxide semiconductor (CMOS) image sensor may be used for the photodetector 12.

The control unit 13 is configured to analyze the change in the traveling direction of the light 19 received by the photodetector 12. In an example, the control unit 13 uses a single-board computer such as a Raspberry Pi® or an Arduino® equipped with an arithmetic unit, such as a CPU (not shown) and a storage device such as a memory (not shown).

The measuring unit 14 acquires intensity information of the light received by each light receiving element of the photodetector 12. The acquired intensity information is transmitted to the control unit 13. In an example, the measuring unit 14 is constructed using a dedicated integrated circuit.

In an example, function of the measurement device 10 is described in detail in conjunction with FIG. 3. The light 18 is emitted from the light source 11 and introduced into the introductory part 3 of the measurement chip 1. The light propagates while getting totally reflected inside the propagation layer 2. An amount of phase shift when the light is totally reflected depends on a magnitude of the refractive index of the surrounding material in contact with the propagation layer 2. On the surface of the propagation layer 2, there are regions where the ligand layer 6 is formed and regions where the ligand layer 6 is not formed. Therefore, on the surface of the propagation layer 2, the amount of phase shift when the light is totally reflected is different between the regions where the ligand layer 6 is formed and the regions where the ligand layer 6 is not formed.

To this end, the light propagating in the Y-axis direction between the introductory part 3 and the outgoing part 4 in the propagation layer 2 changes the phase distribution in the X-axis direction. Therefore, the phase distribution of the light derived from the derivation part 4 is inclined along the X-axis direction, and the traveling direction of the light is deflected. When the ligand 72 in the ligand layer 6 and the analyte 75 in the object to be measured react, the traveling direction of the light changes as the amount of phase shift in the region where the ligand layer 6 is formed changes.

Continuing further, the measurement device 10 receives the light 19 derived from the outgoing part 4 in a far field (or through a Fourier transform lens) using the photodetector 12. Thereafter, the measuring unit 14 measures a change of the angle at which the intensity peaks. The change of the peak angle may be same phenomenon as a change of the traveling direction of the light, and the change of the angle at which the intensity peaks corresponds to the change of the traveling direction of the light. The change of the peak angle measured by the measuring unit 14 is input to the control unit 13 and recorded appropriately in a storage device (memory) in the control unit 13.

In an example, the control unit 13 is equipped with an arithmetic unit (CPU) and determines that the ligand 72 in the ligand layer 6 has reacted with the analyte 75 when the change of the peak angle is, for example, above a predetermined threshold. Alternatively, the control unit 13 estimates the concentration or kinetic parameters of the analyte 75 based on a shape of a plot of the amount of change of the peak angle over time. In this way, the control unit 13 performs analytical processing to analyze the change of the light pattern.

### Measurement Method

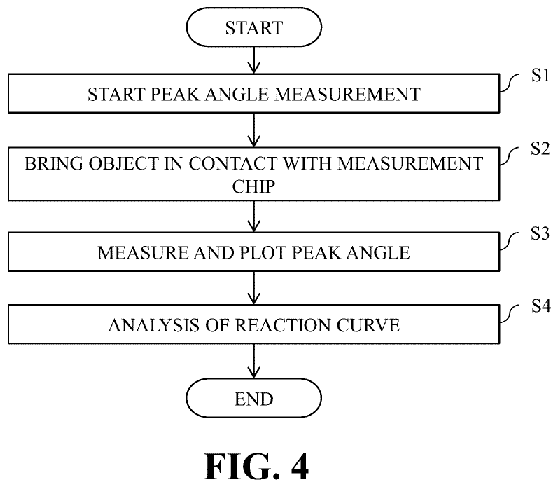

FIG. 4 illustrates a flowchart of a measurement method, according to an embodiment. In an example, the measurement device 10 having the measurement chip 1 is used for one sensing lane for performing the measurement method. For measurement, for example, a member having a concave cross-section is covered over the upper surface of the measurement chip 1, and a flow path is provided between the upper surface of the measurement chip 1 and the member.

At step S1, a measurement of a peak angle is started. In an example, the peak angle is acquired and plotted in real time. On the surface of the propagation layer 2, the ligand layer 6 that reacts with the analyte 75 in the measurement object is formed. The measurement of the peak angle is carried out by introducing the light into the propagation layer 2 through the introductory part 3 of the measurement chip 1. Thereafter, the peak angle of the intensity of the light totally reflected in the propagation layer 2 is measured. The totally reflected light may be derived out from the propagation layer 2 through the outgoing part 4. For example, the amount of change in the peak angle, i.e., the direction in which the light travels, approximately matches a value obtained by dividing a distance between the measurement chip 1 and the photodetector 12 from the amount of change in the peak angle on the photodetector 12.

At step S2, the object to be measured is brought into contact with the measurement chip 1. In an example, the contact of the object with the measurement chip 1 is made by bringing the object containing the analyte 75 into contact with the upper surface of the measurement chip 1. In certain cases, a buffer is brought into contact with the measurement chip 1 before and after the object is brought into contact. For example, bringing the buffer into contact with the measurement chip 1 before it is brought into contact with the object causes to reflect the effects, such as a volume change of the scaffold material, of the refractive index of the object in a measurement signal. In particular, bringing the buffer in contact with the measurement chip 1 after it is brought into contact with the object improves the accuracy of subsequent analysis using the measurement chip 1.

When there are multiple measuring objects, the step S2 may be repeated multiple times. When the step S2 is repeated multiple times, a regeneration process is performed as an optional process. The regeneration process is performed by exposing the measurement chip 1 to an acidic solution with, for example, a pH in a range of 3˜1 to dissociate the ligand 72 and the analyte 75 in a short time. The regeneration process may be omitted if the analyte 75 is rapidly dissociated.

At step S3, measurement and plotting of the peak angle is performed. In an example, the peak angle may be measured and plotted in the direction in which the light travels, i.e., Y-axis direction. The peak angle may be plotted in a reaction curve.

At step S4, analysis of the reaction curve is performed. The reaction curve may indicate a shape of the graph plotting the amount of change in the peak angle. In the analysis, a determination may be made to check if the ligand 72 in the ligand layer 6 has reacted with the analyte 75. In other words, the analysis may be performed to check if the analyte 75 is present in the object to be measured. For example, if the change in the peak angle is above a predetermined threshold, presence of analyte 75 may be determined. Alternatively, the concentration or the kinetic parameters of the analyte 75 may be estimated based on the shape of the graph plotting the change in the peak angle. Thus, according to the measurement method in one embodiment, the presence or absence of the analyte 75 may be determined or the concentration or kinetic parameters of the analyte 75 may be estimated.

In the measurement using the measurement chip 1, the number of times light is reflected inside the propagation layer 2 may be adjusted by changing the length of the measurement chip 1 in the Y-axis direction. Thus, the sensitivity of the measurement chip 1 may be changed. For example, a number of times the light is reflected in the propagation layer may be large if the length in the Y-axis direction is longer, so the sensitivity of the measurement is improved.

In the measurement using the measurement chip 1, even if the output intensity of the light source 11 changes, the amount of change in the peak angle does not change. This enables stable measurement using the measurement chip 1, even if the operation of the light source 11 is somewhat unstable.

As described above, according to the measurement chip 1 according to the first embodiment, it is possible to expand the allowable range of the incident angle, θ, of the light 18 incident on the introductory part 3. According to the measurement device 10 and the measurement method using the measurement chip 1 described in the present embodiment, the allowable range of the incident angle, θ, of the light 18 incident on the introductory part 3 is expanded. This enables to perform the measurement stably even if there is a variation in a thickness of the propagation layer 2 due to a variation in the chip fabrication process or a variation in the wavelength of the light source 11.

### Second Embodiment

The configuration of the measurement chip and/or measurement device in the second to fifth embodiment described below is the same as the configuration of the measurement chip and/or measurement device in the first embodiment unless otherwise noted, and therefore overlapping descriptions are omitted. As long as technical inconsistencies do not arise, the configuration of the measurement chip and/or measurement device in the first to fifth embodiment can be combined as appropriate.

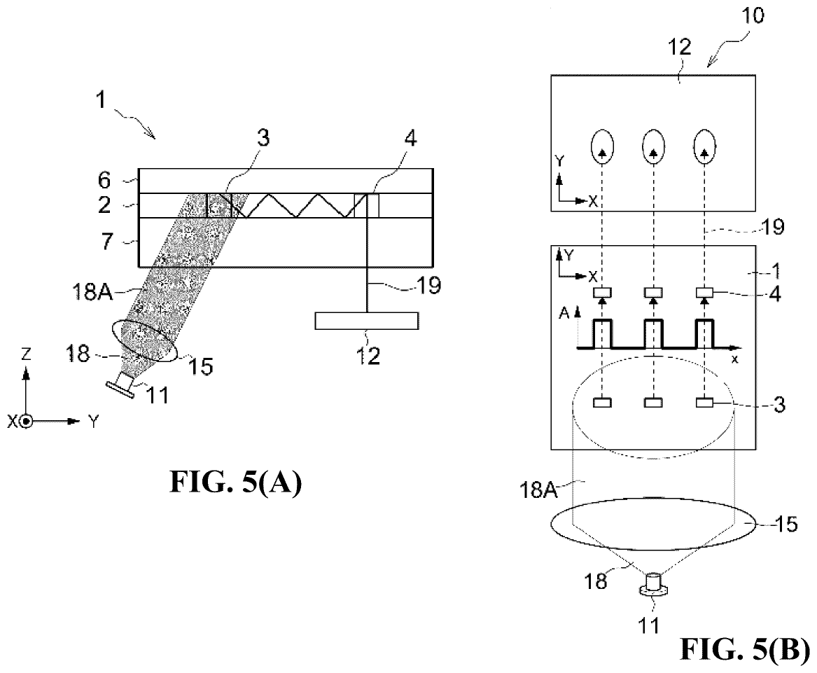

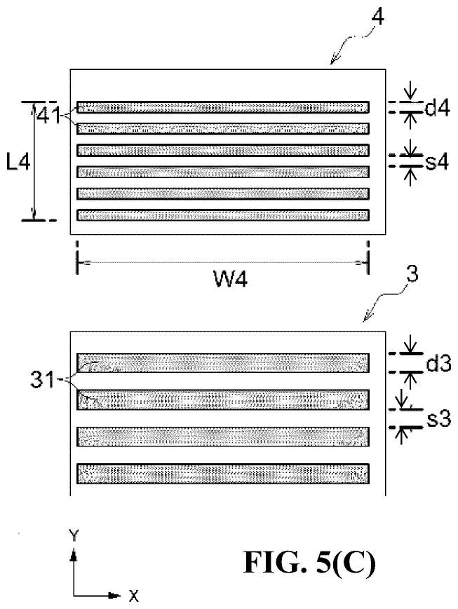

FIGS. 5(A)-5(C) illustrate diagrams showing a schematic structure of the measurement device and the measurement chip, according to another embodiment. FIG. 5(A) shows a side view of the measurement chip 1. FIG. 5(B) shows a schematic configuration of the measurement device 10 having the measurement chip 1 of the present embodiment. FIG. 5(C) is a planar view of a diffraction grating provided at the introductory part 3 and the outgoing part 4 of the measurement chip 1.

According to the present embodiment, the measurement chip 1 includes multiple sets of introductory parts (such as the introductory part 3), multiple sets of propagation layers (such as the propagation layer 2) and multiple sets of outgoing parts (such as the outgoing part 4). The multiple sets of the introductory part 3, the propagation layer 2, the outgoing part 4, and the ligand layer 6 in the direction along the optical axis may be divided substantially into multiple pieces such that the measurement chip 1 includes multiple sensing lanes. In an example, the measuring device 10 may include a collimator lens 15 for outputting the light 18 emitted from the light source 11 as a wide collimated light 18A. The collimator lens 15 may be arranged between the light source 11 and the measurement chip 1. For example, the light 18 emitted by the light source 11 may not be a Gaussian beam.

Referring to FIGS. 5(A) and 5(B), the measuring device 10 using the measurement chip 1 may include the light 18 emitted from the light source 11 that is widely irradiated to include some or all of the multiple introductory parts provided in the measurement chip 1. As a result, since only the light introduced into the propagation layer 2 through the diffraction grating of each of the introductory parts propagate in the propagation layer 2, the light 18 emitted from the light source 11 can be substantially made into plural optical axes without dividing it into plural optical axes using a beam splitter. In addition, a misalignment of the light 18 and the measurement chip 1 can be allowed only by the extent that the width dimension of the widely incident light 18 is longer than a dimension of both ends of the multiple introductory parts arranged in a line on the measurement chip 1. This improves the problem of the accuracy of the alignment of the light 18 and the measurement chip 1.

Referring to FIG. 5(C), an average value of the periods of the grating patterns is different between the first diffraction grating provided in the introductory parts and the second diffraction grating provided in the outgoing part 4.

In the example, the width d3 of the grating patterns 31 of the first diffraction grating provided in the introductory part 3 is larger than a width d4 of grating patterns 41 of the second diffraction grating provided in the outgoing part 4. Moreover, the interval s3 of the grating patterns 31 of the first diffraction grating provided in the introductory part 3 is larger than interval s4 of the grating patterns 41 of the second diffraction grating provided in the outgoing part 4. This prevents interference in the photodetector 12 between the light derived from the outgoing part 4 and the light reflected on the surface of the measurement chip 1.

The periods of the grating patterns 31 of the first diffraction grating provided in the introductory part 3, i.e., the sum of the width d3 and the interval s3, and periods of the grating patterns 41 of the second diffraction grating provided in the outgoing part 4, i.e., a sum of the width d4 and the interval s4, are set so that an incident angle of the light source 11 and an exit angle of the light emitted from the propagation layer 2 are different to some extent in order to sufficiently prevent interference. For example, the period of the grating patterns 31 may be, for example, about 470 nm; and the period of the grating patterns 41 may be, for example, about 320 nm. In such a case, assuming that the wavelength of the light is 520 nm, the incident angle may be, for example, 30 degrees; and the exit angle may be, for example, 0 degrees. For the grating patterns 31 of the first diffraction grating provided in the introductory part 3, as illustrated in FIG. 1(B), periods of the multiple grating patterns 31 of the first diffraction grating in the Y-axis direction is different from each other between the two or more regions, such as the region 1, the region 2 and the region 3.

As shown in FIG. 5(C), the width d4 of the multiple grating patterns 41 and the spacing s4 of the multiple grating patterns 41 are constant along the direction in which light propagates, i.e., the Y-axis direction, in the second diffraction grating provided in the outgoing part 4. For example, for the outgoing part 4, a length L4 in the Y-axis direction is about 100 microns and a length W4 in the X-axis direction is about 550 microns.

### Third Embodiment

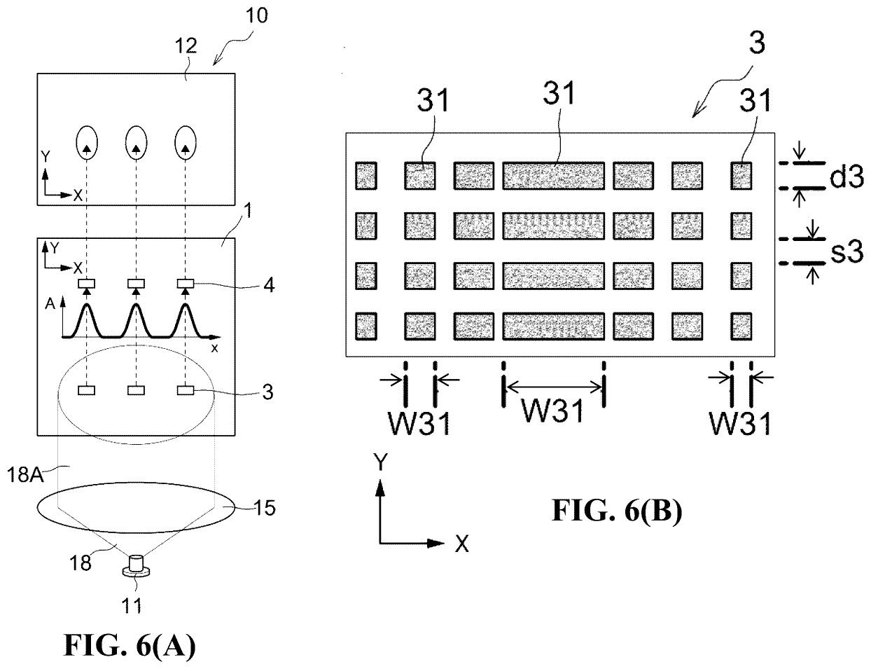

FIGS. 6(A) and 6(B) illustrate diagrams showing a schematic structure of the measurement device and the measurement chip, according to yet another embodiment of the present disclosure. FIG. 6(A) illustrates a diagram showing the schematic structure of the measurement device 10. FIG. 6(B) is a planar view of the first diffraction grating provided at the introductory part 3 of the measurement chip 1.

Referring to FIG. 6(B), the diffraction grating provided at the introductory part 3 has a planar shape of the grating patterns 31 with reduced coupling efficiency at both ends of the perpendicular direction, i.e., the X-axis direction, to the light propagation direction, i.e., the Y-axis direction. This may controls a complex amplitude distribution of the light introduced into the propagation layer 2 from the introductory part 3.

In the example shown, a width W31 of the grating patterns 31 in the X-axis direction may decrease from a center of the introductory part 3 towards both the ends. Therefore, if the first diffraction grating of the introductory part 3 has such a planar shape of the grating patterns 31 causing the coupling efficiency to decrease at both ends, the light 18 introduced into the propagation layer 2 from the introductory part 3 will have a rectangular amplitude distribution with a smaller amplitude distribution at both the ends. As the amplitude distribution of the light becomes smaller at both the ends, a sidelobe intensity at the far field becomes smaller, thus reducing interference at the photodetector 12.

In an example, if a shape of the first diffraction grating provided at the introductory part 3 along the X-axis direction is a single rectangle, as illustrated in FIG. 5(C), then the amplitude distribution of the light on the photodetector 12 becomes a SINC function, and if the distance between adjacent sensing grains is close, interference occurs between them.

### Fourth Embodiment

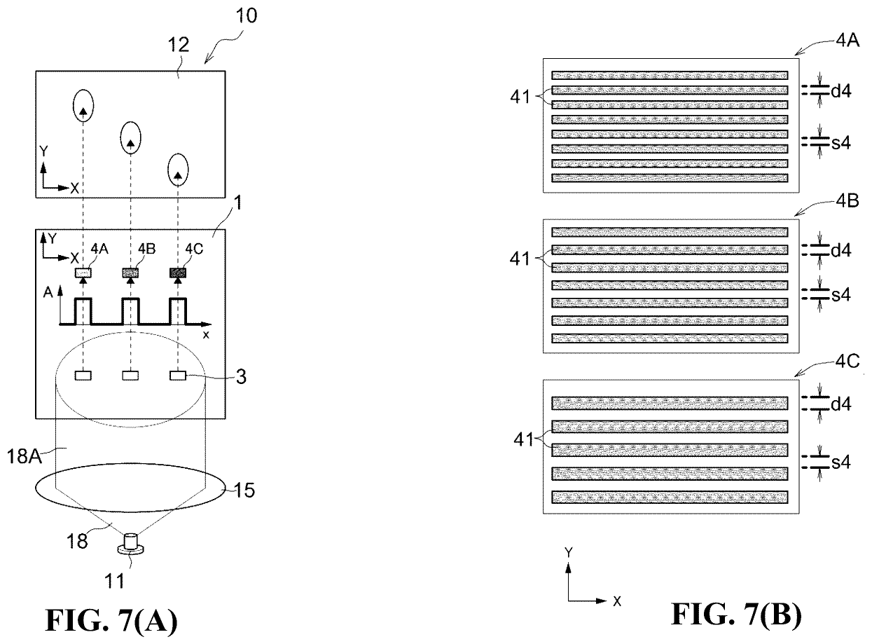

FIGS. 7(A) and 7(B) illustrate diagrams showing the schematic structure of the measurement device 10 and the measurement chip 1, according to another embodiment of the present disclosure. FIG. 7(A) is a diagram showing the schematic configuration of the measuring device 10. FIG. 7(B) is a planar view of the second diffraction grating provided at the outgoing part 4 of the measurement chip 1.

Referring to FIG. 7(A) and FIG. 7(B), the second diffraction grating provided at the outgoing parts 4A, 4B and 4C differ among multiple sensing grains along the width d4 in the Y-axis direction of the multiple grating patterns 41 or along the spacing s4 of the grating patterns 41, or along a combination of the width d4 and the spacing s4. In other words, the second diffraction grating provided at the outgoing part 4A, 4B, 4C differs among the multiple sensing grains among the multiple sets in period of the multiple grating patterns 41. Thus, positions, such as the Y-axis direction of the light, derived from the outgoing part 4A, 4B, 4C on the photodetector 12 are different from each other, so that interference in the photodetector 12 is reduced.

### Fifth Embodiment

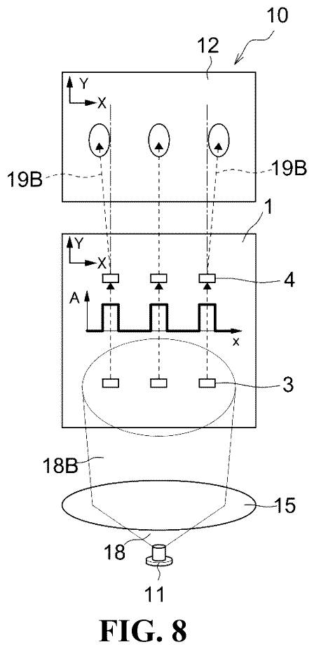

FIG. 8 is a diagram showing the schematic structure of the measuring device 10, according to an embodiment.

In an example, the measuring device 10 may include the collimate lens 15 such that the collimate lens 15 is arranged in an optical system that is out of collimate condition. For example, a distance between the light source 11 and the collimate lens 15 is adjusted by moving a position of the collimate lens 15 along an optical axis toward the light source 11 so that a broad light 18B is irradiated on the introductory part 3 of the measurement chip 1. The broad light 18B is out of the collimate condition. Thus, in the measuring device 10, the positions in the X-axis transverse direction on the photodetector 12 may be separated from each other, for example, by deviating the optical axis of the light derived from the outgoing part 4 by more than a distance between sensing grains, thereby reducing interference in the photodetector 12.

Conversely, when the position of the collimator lens 15 is moved along the optical axis toward the measurement chip 1, the optical axes of the light derived from the outgoing part 4 may be brought closer to each other, and more output light can be observed simultaneously. Thus, using the measuring device 10, it becomes possible to adjust the position of the optical axis 19B between adjacent sensing grains according to the size of the photodetector 12 used for measurement.

Other ways to remove the collimating lens 15 from the collimating condition include, for example, adjusting a focal length of the collimating lens 15 while maintaining a positional relationship between the light source 11 and the collimating lens 15 along the optical axis.

### Other Forms

While the present invention has been described above in terms of specific embodiment(s), the present invention is not limited to the above-mentioned embodiment(s).

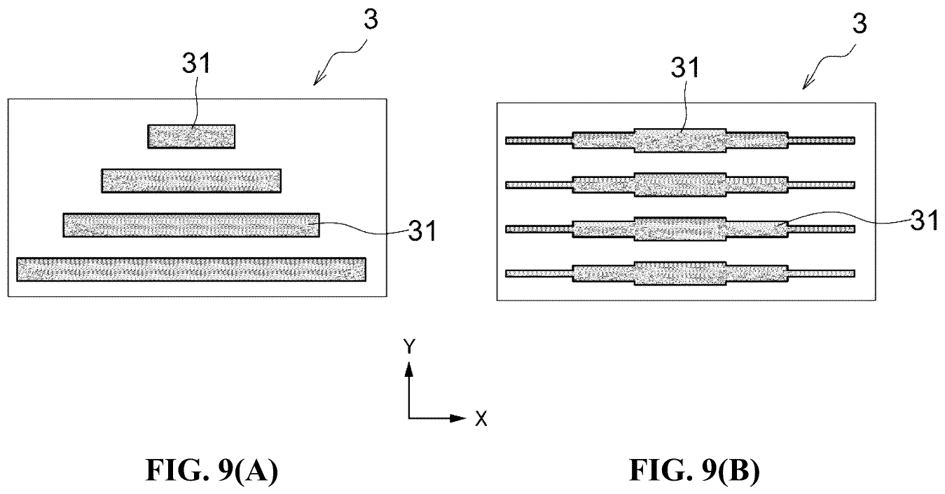

FIGS. 9(A) and 9(B) illustrate planar views showing a variation of the first diffraction grating provided at the introductory part 2 of the measurement chip 1, according to an embodiment. FIGS. 9(A) and 9(B) show variations in a planar shape of the grating patterns 31 with reduced coupling efficiency at both ends, as illustrated in FIG. 6(B).

In the above third embodiment, the planar shape shown in FIG. 6(B) is exemplified as the planar shape of the first diffraction grating provided at the introductory part 3 for controlling the complex amplitude distribution of light introduced into the propagation layer 2 from the introductory part 3, but the planar shape of the first diffraction grating provided at the introductory part 3 is not limited to the planar shape shown in FIG. 6(B). In order to control the complex amplitude distribution of the light introduced into the propagation layer 2 from the introductory part 3, the planar shape of the grating pattern 31 with reduced coupling efficiency at both ends of the perpendicular direction (X-axis direction in the figure) to the light propagation direction (Y-axis direction in the figure) can be the planar shape shown in FIG. 9(A) or the planar shape shown in FIG. 9(B).

FIGS. 9(A) and 9(B) illustrate planar views showing a variation of diffraction grating provided at the measurement chip 1, according to an embodiment.

In the above embodiment, examples of antigens and antibodies are shown as combinations of analytes and ligands, but combinations are not limited to this. As combinations of analytes and ligands, enzymes and substrates, hormones and receptors, DNA complements, etc. are also possible. Even in these cases, on the surface of the propagation layer 2, the amount of phase shift in total reflection of light differs between a region where the ligand layer 6 is formed and a region where the ligand layer 6 is not formed, and it goes without saying that the amount of phase shift in the region where the ligand layer 6 is formed is changed by the combination of the analytes and the ligands.

In the measurement device 10 and the measurement method using the measurement chip 1 according to the above embodiment, a binding reaction of a biomolecule is used as an example, and should not be construed as a limitation. The measurement device 10 and the measurement method using the measurement chip 1 may be applied if the reaction involves a refractive index change even if it is not the binding reaction of the biomolecule to be exemplified. As an example, the measuring device 10 and measurement method using the measuring chip 1 according to the above embodiment can be applied to a gas sensor, etc. In this case, the gas can be used as an analyte, and a chemical substance whose refractive index changes when it reacts with the gas can be used as a ligand.

In the above embodiment, the introductory part 3 of the measurement chip 1 is provided with diffraction gratings that differ from each other in regions where the periods of the multiple grating patterns 31 are two or more, however, such diffraction gratings may also be provided in the outgoing part 4. This allows the light derived from the outgoing part 4 to retain the intensity of the light over a wide angular range, thereby, improving the problem of the alignment of the CCD image sensor. In addition, in the measurement chip 1 composed of multiple sensing lanes, a diffraction grating with a constant period of the grating pattern may be provided in the introductory part 3 in some sensing lanes, and a diffraction grating with an increase or decrease in the period of the grating pattern may be provided in the outgoing part 4 in other sensing lanes. In particular, in the measurement chip 1, the periods of the multiple grating patterns provided in each diffraction grating may differ from each other between two or more regions in either the first diffraction grating provided in the introductory part 3 or the second diffraction grating provided in the outgoing part 4.

For the light 18 incident on the introductory part 3 of the measurement chip 1, the light having an electric field in the Z-axis direction, as shown in the FIG. 1(A), is defined as P-polarized light, and the light having an electric field in the X-axis direction is defined as S-polarized light. Although P-polarized light is incident on the introductory part 3 in the above embodiment, the light 18 incident on the introductory part 3 may be either P-polarized or S-polarized. The effect of the present invention that it is possible to expand the allowable range of the incident angle, θ, of the light 18 incident on the introductory part 3 can be achieved when the incident light is any of P-polarized or S-polarized. However, the effect of the present invention is more pronounced when the incident light is P-polarized. The reason is that when the incident light is P-polarized, the allowable range of the incident angle, θ, is very narrow at about 0.6 degrees as shown in the conventional technique, while when the incident light is S-polarized, the allowable range is about 1.5 degrees to 2 degrees, which is somewhat wider from the original as compared with the case of P-polarized light. It may be noted that when the incident light is P-polarized, the sensitivity, i.e., the amount of change in the peak angle or the amount of change in the refractive index, is higher than when the incident light is S-polarized, and the coupling efficiency to the propagation layer 2 is also higher, so the practicality of P-polarized light is higher than that of S-polarized light.

### EXAMPLE

In an example, an allowable range of the incident angle, θ, of the light entering the introductory part 2 is verified by actual measurement. The verification may be carried out by preparing multiple measurement chips in which diffraction gratings with different periods of grating patterns for each region along the direction in which the light propagates were provided. Further, intensity of the light derived from the outgoing part 4 may be measured while varying the incident angle, θ, of the light entering the introductory part 3 for each of the prepared multiple measurement chips. Light having an electric field may be injected into the introductory part 3 in the thickness direction of the measurement chip, i.e., the Z axis direction.

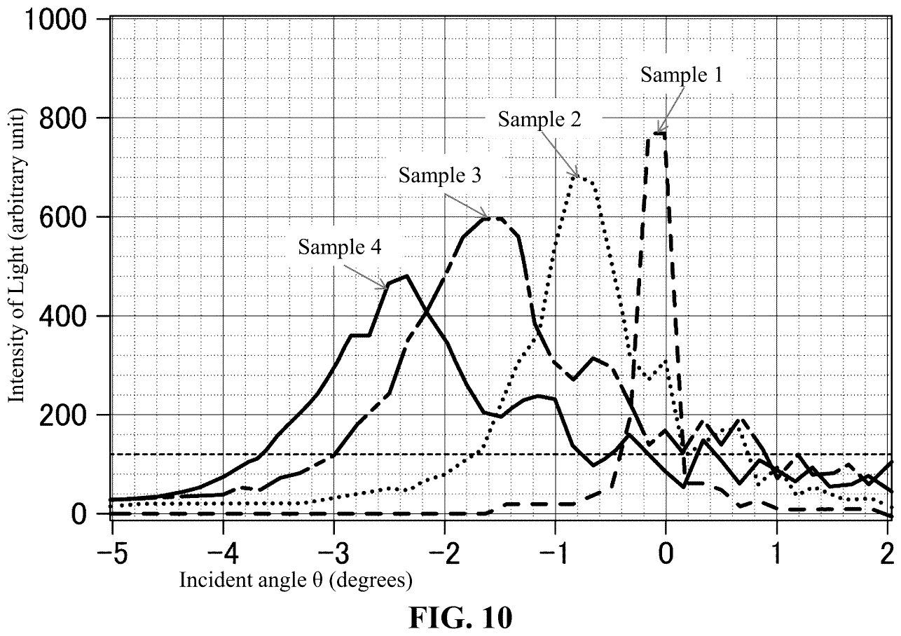

Variations of the diffraction grating provided at the introductory part 3 of the measuring chip are shown in Table 1. A graph of the measurement results is shown in FIG. 10. In particular, FIG. 10 is a graph showing allowable range of the incident angle, θ, of the light, according to an embodiment. In an example, the period of the grating pattern is the period (d3+s3) in the Y-axis direction for the grating patterns 31, and the incident angle θ of the light is the angle θ shown in FIG. 1(A). For example, the wavelength of the light used for the measurement is 520 nm.

TABLE 1

| | grating | grating | grating | Number of |
|---|---|---|---|---|
| | minimum | central | Maximum | periodic |
| Sample | period | period | period | divisions |
| Number | [nm] | [nm] | [nm] | (Every 2.5 nm) |
| 1 | 472.5 | 472.5 | 472.5 | 1 |
| 2 | 465.0 | 472.5 | 480.0 | 7 |
| 3 | 457.5 | 472.5 | 487.5 | 13 |
| 4 | 450.0 | 472.5 | 495.0 | 19 |

For the region of the introductory part 3 where the length L3 in the Y-axis direction is about 100 microns and the length W3 in the X-axis direction is about 270 microns, each periodic part may be created by dividing the length L3 in the Y-axis direction by the number of periodic divisions shown in Table 1.

For the dimension of each periodic part in the Y-axis direction for one period, the width of the grating pattern and the spacing of the grating pattern may be made to have a same dimension. The dimension of one period in the Y-axis direction means the sum of the width of the adjacent grating pattern along the Y-axis direction and the spacing of the grating pattern, that is, the period. In an example, a first diffraction grating of a sample number 1 may be a single-period grating pattern with a period division number of 1. In such a case, all the Y-axis dimensions for one period may be 472.5 nm. In another example, in the case of the measurement chip of sample number 2, a first diffraction grating for the introductory part 3 may have the following grating pattern.

The Y-axis dimension of the 465.0 nm periodic part may be about 14.3 microns.

The Y-axis dimension of the 467.5 nm periodic part may be about 14.3 microns.

The Y-axis dimension of the 470.0 nm periodic part may be about 14.3 microns.

The Y-axis dimension of the 472.5 nm periodic part may be about 14.3 microns.

The Y-axis dimension of the 475.0 nm periodic part may be about 14.3 microns.

The Y-axis dimension of the 477.5 nm periodic part may be about 14.3 microns.

The Y-axis dimension of the 480.0 nm periodic part may be about 14.3 microns.

Referring to FIG. 10, a horizontal axis of the graph shows the angle of incidence, θ. Moreover, a point where the angle of incidence, θ, is 0 degrees in FIG. 10 corresponds to a point where the angle of incidence, θ, is about 30 degrees in FIG. 1(C). Further, a vertical axis of the graph shows an emission intensity of light. As shown, a line of a lowest intensity of light that can be sufficiently measured by a receiver such as a CCD image sensor corresponds to a line where an emission intensity is about 120 (arbitrary unit).

### Range of Incident Angle That can be Measured

A range of the incident angle, θ, that can be measured, is discussed with reference to FIG. 10. The range of the incident angle that can be measured may be, for example, 0.6 degrees (such as between −0.4 degrees to +0.2 degrees) when the grating pattern of the first diffraction grating provided at the introductory part 3 is single period as shown in the dashed line graph for sample number 1. On the other hand, when the period of the grating pattern of the first diffraction grating provided at the introductory part 3 is different from region to region along the direction of light propagation as shown in sample number 2 to sample number 4, the range of the incident angle, θ, that can be measured is larger than that of sample number 1 for the sample number 2, sample number 3 and sample number 4.

For example, the range of the incident angle, θ, that can be measured is about 4.0 degrees (−3.0 degrees to +1.0 degrees) in the case of the measurement chip 1 of sample number 3 as shown in the graph shown by the dashed line. For example, as shown in the dotted line graph, in the case of the measurement chip 1 of the sample number 2, the range of the incident angle, θ, that can be measured may be about 2.6 degrees (−1.8 degrees to +0.8 degrees). For example, as shown in the solid line graph, in the case of the measurement chip 1 of sample number 4, the range of the incident angle, θ, that can be measured may be about 3.4 degrees (−3.6 degrees to −0.2 degrees). It may be noted that the range of the incident angle, θ, that can be measured can be expanded if a diffraction grating whose period of the grating pattern varies from region to region along the direction in which light propagates is provided in the introductory part 3.

As shown in Table 1, a central value of the grating period may be 472.5 nm for each of the sample numbers 1 to 4. However, in the graph of the FIG. 10, the value of the incident angle, θ, i.e., the center of the incident angle at which emission intensity peaked is deviated from 0 degrees for each of the sample numbers 2, 3 and 4.

### Displacement of the Incident Angle Theta

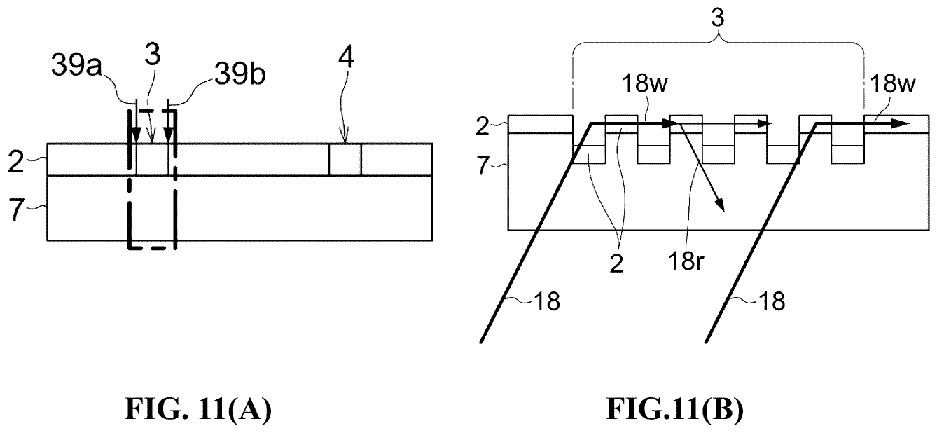

FIGS. 11(A) and 11(B) illustrate diagrams showing the schematic structure of the measurement chip 1 indicating deviation of peak angle of the incident angle, θ. of the light, according to an embodiment. FIG. 11(A) is a side view of the measurement chip 1. FIG. 11(B) is a partially enlarged view of an area enclosed by dash-dot lines in FIG. 11(A).

The graph in FIG. 10 is considered in terms of a point where a value of the peak angle of incidence, θ, deviates. Pursuant to present disclosure, the dimensions of the first diffraction grating provided at the introductory part 3 along the direction of light propagation that may be approximately 100 microns may have grating patterns. The grating patterns in all regions of the first diffraction grating may not uniformly contribute to the coupling with the propagation layer 2. In addition, within the introductory part 3, a region 39a is present; and a region 39b is present near the outgoing part 4 from which the light is taken out. For example, the region 39b is considered to have a larger contribution of the coupling to the propagation layer 2.

As shown in FIG. 11(B), when the light 18 is irradiated to the introductory part 3 and introduced into the propagation layer 2 through the introductory part 3, light 18w propagates in a guided mode through the propagation layer 2. At this time, some of the light 18w becomes light 18r in a radiation mode by the first diffraction grating of the introductory part 3 and is emitted from the propagation layer 2. Therefore, a consideration may be made that the light 18w in the guided mode is unlikely to become the radiation mode again because the region 39b of the introductory part 3, which is closer to the outgoing part 4 from which the light is taken out, has a shorter distance to interact with the first diffraction grating of the introductory part 3.

Further, the above consideration also yields that rather than dividing the regions of each period into equal intervals, as shown in Table 1, it may be possible to widen the region where the contribution of the coupling to the propagation layer 2 is small and narrow the region where the contribution of the coupling to the propagation layer 2 is large so that the range of the incident angle, θ, that can be measured can be widened. For example, in the diffraction grating of sample number 2, in Table 1, the region 39a side is 465.0 nm and the region 39b side is 480.0 nm, as shown in FIG. 11(A). In such a case, a size in the Y-axis direction of the light having the wavelength 465.0 nm may have a periodic part larger than, for example, 14.3 micrometers. Further, the size in the Y-axis direction of the light having the wavelength 480.0 nm may have a periodic part smaller than 14.3 micrometers.

The above description describes the embodiments of the present disclosure. Many modifications and other embodiments of the disclosures set forth herein will come to mind to one skilled in the art to which these disclosures pertain having the benefit of the teachings presented in the foregoing descriptions and the associated drawings. Therefore, it is to be understood that the disclosures are not to be limited to the specific embodiments disclosed and that modifications and other embodiments are intended to be included within the scope of the appended claims. Moreover, although the foregoing descriptions and the associated drawings describe example embodiments in the context of certain example combinations of elements and/or functions, it should be appreciated that different combinations of elements and/or functions may be provided by alternative embodiments without departing from the scope of the appended claims. In this regard, for example, different combinations of elements and/or functions than those explicitly described above are also contemplated as may be set forth in some of the appended claims. Although specific terms are employed herein, they are used in a generic and descriptive sense only and not for purposes of limitation.

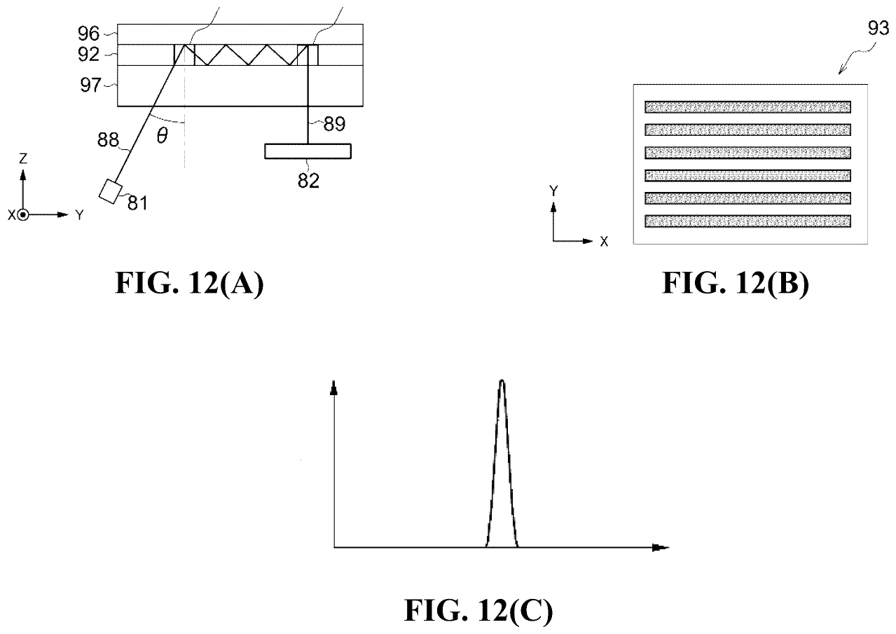

FIGS. 12(A)-12(C) illustrates diagrams showing a schematic structure of a conventional optical waveguide type measurement chip 91, according to an embodiment. FIG. 12(A) is a side view of the conventional measurement chip 91. FIG. 12(B) is a top view of a diffraction grating provided at an introductory part 93 of the conventional measurement chip 91. FIG. 12(C) is a view showing a relationship between an intensity of light derived from the introduction and an incident angle of light in the conventional measurement chip.

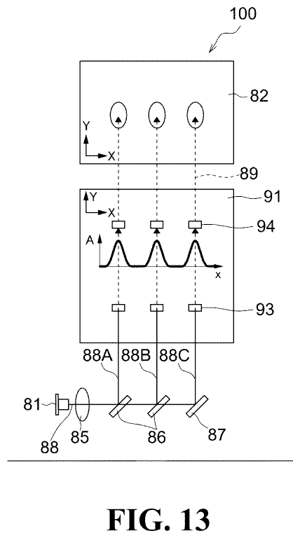

FIG. 13 illustrates a diagram showing the schematic structure of a conventional measurement device 100 when multiple measurement objects are measured in parallel using the conventional optical waveguide type measurement chip 91, according to an embodiment.

As illustrated in FIG. 12(A), the conventional optical waveguide type measurement chip 91 includes with a propagation layer 92 where light propagates, an introductory part 93 which introduces light into the propagation layer 92, an outgoing part 94 which derives light from the propagation layer 92, and a ligand layer 96. The ligand layer 96 is formed when, or before, the measurement chip 91 is used for measurement by modifying the surface of the propagation layer 92 with a ligand, such as antibodies that reacts with an analyte, such as antigen in a measuring object (e.g., a specimen). A transparent substrate 97 is provided on a lower surface of the propagation layer 92.

When the measurement chip 91 is used for measurement, light 88 is emitted from the light source 81 and introduced into the propagation layer 92 from the underside of the measurement chip 91 through the introductory part 93. The introductory part 93 uses a diffraction grating having a grating pattern shown in the FIG. 12(B). The light totally reflected inside the propagation layer 92 is derived from the lower surface of the measurement chip 91 through the outgoing part 94 and is received by the photodetector 82.

In an example, a first requirement for measurement stability is to expand an allowable range of the incident angle, θ, of the light incident on the introductory part 93. As shown in FIG. 12(C), the conventional measurement chip 91 has a problem that the allowable range of the incident angle of the light 88 incident on the introductory part 93 (approximately 0.6 degrees, for example) is narrow, and if the incident angle of the light 88 deviates from the optimum angle, a guided wave efficiency greatly decreases. Since an optimum incident angle of the light 88 varies depending on the variation in the thickness of the propagation layer 92 constituting the waveguide and the variation in the wavelength of the light 88 emitted from the light source 81, there is a problem that the measurement becomes impossible when these conditions regarding the thickness of the propagation layer 92 and the wavelength of the light 88 change. For example, if the thickness of the propagation layer 92 varies in the range of, for example, +/−5 nm due to variation in the process conditions during film formation, the optimum incident angle may have a variation of, for example, +/−1.2 degrees. Furthermore, for example, if a temperature of a semiconductor laser used as the light source 81 varies between 5 and 40 degrees C., the wavelength emitted from the light source 81 varies in the range of, for example, +/−5.25 nm, and if a variation in the thickness of the propagation layer 92 is combined with this variation, the optimum incident angle has a variation of about, for example, +/−2.0 degrees.

Further, a second requirement for measurement stability is to improve a problem of the accuracy of an alignment of the measurement chip 91 with optical axes, such as optical axes 88 A, 88B, and 88 C that are divided into multiple pieces. As shown in FIG. 13, the light 88 emitted from the light source 81 is output as collimated light by a collimated lens 85, then divided into multiple optical axes 88 A, 88B, and 88 C by a beam splitter 86, and injected into the respective introductory parts 94 of the measurement chip 91. In order to process multiple measuring objects in parallel, one measurement chip 91 is provided with multiple sets of introductory parts 94 and outgoing parts 94. The sets of introductory parts 94, propagation layers 92, outgoing parts 94, and ligand layers 96 in the directions along the respective divided optical axes 88 A, 88B, 88 C may include multiple sensing lanes on one measurement chip 91. Thus, in the conventional measuring device 100, the light 88 from the light source 81 is divided into multiple optical axes 88A, 88B, and 88C by the beam splitter 86 in order to process the multiple objects in parallel. As a result, there is a problem that high accuracy is required for the alignment with the outgoing parts 94 for the respective optical axes 88A, 88B, and 88C. When the accuracy of the alignment between the optical axes 88 A, 88B, and 88 C divided into the multiple axes and the measurement chip 91 decreases, there may arise a problem that the intensity of the light derived from the outgoing part 94 decreases or the beam shape deforms, making it impossible to make accurate measurements.

Embodiments of the present disclosure, as described in conjunction with FIGS. 1-11 aim to overcome the aforementioned drawbacks associated with the conventional optical waveguide-type measurement chips. The present disclosure provides measurement chips that enable accurate and reliable measurement of presence or absence, concentration and/or kinematics of an analyte using diffraction gratings having periods of grating patterns that may be different from each other in different regions.

It is to be understood that not necessarily all objectives or advantages may be achieved in accordance with any particular embodiment described herein. Thus, for example, those skilled in the art will appreciate that certain embodiments may be configured to operate in a manner that achieves or optimizes one advantage or group of advantages as taught herein without necessarily achieving other objects or advantages as may be taught or suggested herein.

All processes described herein may be embodied in, and fully automated via, software code modules executed by a computing system that includes one or more computers or processors. The software code modules may be stored in any type of non-transitory computer-readable medium or other computer storage device. Some or all methods may be embodied in specialized computer hardware.

Many other variations other than those described herein will be apparent from this disclosure. For example, depending on the embodiment, certain actions, events, or functions of any of the algorithms described herein may be performed in different sequences, and may be added, merged, or excluded altogether (e.g., not all described actions or events are required to execute the algorithm). Moreover, in certain embodiments, operations or events are performed in parallel, for example, through multithreading, interrupt handling, or through multiple processors or processor cores, or on other parallel architectures, rather than sequentially. In addition, different tasks or processes can be performed by different machines and/or computing systems that can work together.

The various exemplary logical blocks and modules described in connection with the embodiments disclosed herein can be implemented or executed by a machine such as a processor. The processor may be a microprocessor, but alternatively, the processor may be a controller, a microcontroller, or a state machine, or a combination thereof. The processor can include an electrical circuit configured to process computer executable instructions. In another embodiment, the processor includes an application specific integrated circuit (ASIC), a field programmable gate array (FPGA), or other programmable device that performs logical operations without processing computer executable instructions. The processor can also be implemented as a combination of computing devices, e.g., a combination of a digital signal processor (DSP) and a microprocessor, a plurality of microprocessors, one or more microprocessors in conjunction with a DSP core, or any other such configuration. Although described herein primarily with respect to digital technology, the processor may also include primarily analog components. For example, some or all of the signal processing algorithms described herein may be implemented by analog circuitry or mixed analog and digital circuitry. A computing environment may include any type of computer system, including, but not limited to, a computer system that is based on a microprocessor, mainframe computer, a digital signal processor, a portable computing device, a device controller, or a computing engine within the device.

Unless otherwise stated, conditional languages such as “can,” “could,” “will,” “might,” or “may” are understood within the context as used in general to convey that certain embodiments include, while other embodiments do not include, certain features, elements and/or steps. Thus, such conditional languages are not generally intended to imply that features, elements and/or steps are in any way required for one or more embodiments or that one or more embodiments necessarily include logic for deciding, with or without user input or prompting, whether these features, elements and/or steps are included or are to be performed in any particular embodiment.

Disjunctive languages, such as the phrase “at least one of X, Y, or Z,” unless specifically stated otherwise, is understood with the context as used in general to present that an item, term, etc., may be either X, Y, or Z, or any combination thereof (e.g., X, Y, and/or Z). Thus, such a disjunctive language is not generally intended to, and should not, imply that certain embodiments require at least one of X, at least one of Y, or at least one of Z to each be present.

Any process descriptions, elements, or blocks in the flow diagrams described herein and/or shown in the accompanying drawings should be understood as potentially representing modules, segments, or parts of code, including one or more executable instructions for implementing a particular logical function or elements in the process. Alternate implementations are included within the scope of the embodiments described herein in which elements or functions may be deleted, executed out of order from that shown, or discussed, including substantially concurrently or in reverse order, depending on the functionality involved as would be understood by those skilled in the art.

Unless otherwise explicitly stated, articles such as “a” or “an” should generally be interpreted to include one or more described items. Accordingly, phrases such as “a device configured to” are intended to include one or more recited devices. Such one or more recited devices can also be collectively configured to carry out the stated recitations. For example, “a processor configured to carry out recitations A, B and C” can include a first processor configured to carry out recitation A working in conjunction with a second processor configured to carry out recitations B and C. The same holds true for the use of definite articles used to introduce embodiment recitations. In addition, even if a specific number of an introduced embodiment recitation is explicitly recited, those skilled in the art will recognize that such recitation should typically be interpreted to mean at least the recited number (e.g., the bare recitation of “two recitations,” without other modifiers, typically means at least two recitations, or two or more recitations).

It will be understood by those within the art that, in general, terms used herein, are generally intended as “open” terms (e.g., the term “including” should be interpreted as “including but not limited to,” the term “having” should be interpreted as “having at least,” the term “includes” should be interpreted as “includes but is not limited to,” etc.).

For expository purposes, the term “horizontal” as used herein is defined as a plane parallel to the plane or surface of the floor of the area in which the system being described is used or the method being described is performed, regardless of its orientation. The term “floor” can be interchanged with the term “ground” or “water surface”. The term “vertical” refers to a direction perpendicular to the horizontal as just defined. Terms such as “above,” “below,” “bottom,” “top,” “side,” “higher,” “lower,” “upper,” “over,” and “under” are defined with respect to the horizontal plane.

As used herein, the terms “attached,” “connected,” “coupled,” and other such relational terms should be construed, unless otherwise noted, to include removable, moveable, fixed, adjustable, and/or releasable connections or attachments. The connections/attachments can include direct connections and/or connections having intermediate structure between the two components discussed.

Numbers preceded by a term such as “approximately,” “about,” and “substantially” as used herein include the recited numbers, and also represent an amount close to the stated amount that still performs a desired function or achieves a desired result. For example, the terms “approximately,” “about,” and “substantially” may refer to an amount that is within less than 10% of the stated amount. Features of embodiments disclosed herein preceded by a term such as “approximately,” “about,” and “substantially” as used herein represent the feature with some variability that still performs a desired function or achieves a desired result for that feature.

It should be emphasized that many variations and modifications may be made t o the above-described embodiments, the elements of which are to be understood as being among other acceptable examples. All such modifications and variations are intended to be included herein within the scope of this disclosure and protected by the following claims.

What is claimed is:

1. A measurement chip, comprising:
a propagation layer configured to allow light to propagate;
an introductory part configured to have a first diffraction grating for introducing the light into the propagation layer;
an outgoing part configured to have a second diffraction grating for deriving the light from the propagation layer; and
a ligand modification surface configured to be a surface of the propagation layer and capable of modifying a ligand that reacts with an analyte be detected, wherein
a period of a plurality of grating patterns formed in at least one of: the first diffraction grating, or the second diffraction grating, is different from each other between two or more regions.

2. The measurement chip as claimed in claim 1, wherein:
the periods of the plurality of grating patterns formed in the first diffraction grating are different from each other between two or more regions along a propagation direction of the light.

3. The measurement chip as claimed in claim 2, wherein:
the first diffraction grating is further configured to have the plurality of grating patterns in which the periods increases or decreases for each of the region along the propagation direction of the light.

4. The measurement chip as claimed in claim 2, wherein:
the first diffraction grating is further configured to have the plurality of grating patterns in which the period increases or decreases for each region along the propagation direction of the light while maintaining a constant duty ratio.

5. The measurement chip as claimed in claim 2, wherein:
the period of the plurality of grating patterns formed in the second diffraction grating is constant along the propagation direction of the light.

6. The measurement chip as claimed in claim 1, wherein:
an average value of the periods of the plurality of grating patterns of the first diffraction grating is different from an average value of the periods of the plurality of grating patterns of the second diffraction grating.

7. The measurement chip as claimed in claim 2, wherein:
the measurement chip is further configured to include a plurality of sets of each of: the introductory part, the propagation layer, the outgoing part and the ligand modification surface on which the ligand is modified on one of measurement chips, and wherein:
the periods of the plurality of grating patterns formed on the second diffraction grating are different among the plurality of sets.

8. The measurement chip as claimed in claim 7, wherein:
the first diffraction grating is further configured to have a planar shape of the plurality of grating patterns with reducing coupling efficiency at both ends in a direction perpendicular to the propagation direction of the light.

9. The measurement chip as claimed in claim 1, wherein:
a phase distribution of the light changes due to a change in a refractive index around the propagation layer caused by a reaction between the analyte and the ligand.

10. A measuring device, comprising:
a measurement chip comprising a propagation layer configured to allow light to propagate,
an introductory part configured to have a first diffraction grating for introducing the light into the propagation layer,
an outgoing part configured to have a second diffraction grating for deriving the light from the propagation layer, and
a ligand modification surface configured to be a surface of the propagation layer and capable of modifying a ligand that reacts with an analyte be detected, wherein:
a period of a plurality of grating patterns formed in at least one of: the first diffraction grating, or the second diffraction grating, is different from each other between two or more regions;
a light source configured to guide the light to the introductory part of the measuring chip;
a photodetector configured to receive the light derived from the outgoing part of the measurement chip; and
processing circuitry configured to analyze a change in a pattern of the light received by the photodetector.

11. The measuring device as claimed in claim 10, wherein:
the processing circuitry is further configured to analyze a change in a traveling direction of the light.

12. The measuring device as claimed in claim 10, further comprising:
a collimator lens configured to be positioned between the light source and the introductory part, and collimate light emitted from the light source to illuminate a plurality of introductory parts.

13. The measuring device as claimed in claim 12, wherein:
the collimator lens is further configured to be positioned in an optical system that is out of collimate condition.

14. The measurement chip as claimed in claim 3, wherein:
the first diffraction grating is further configured to have the plurality of grating patterns in which the period increases or decreases for each region along the propagation direction of the light while maintaining a constant duty ratio.

15. The measurement chip as claimed in claim 14, wherein:
the period of the plurality of grating patterns formed in the second diffraction grating is constant along the propagation direction of the light.

16. The measurement chip as claimed in claim 15, wherein:
an average value of the periods of the plurality of grating patterns of the first diffraction grating is different from the an average value of the periods of the plurality of grating patterns of the second diffraction grating.

17. The measurement chip as claimed in claim 16, wherein:
the measurement chip is further configured to include a plurality of sets of each of: the introductory part, the propagation layer, the outgoing part and the ligand modification surface on which the ligand is modified on one of measurement chips, and wherein:
the periods of the plurality of grating patterns formed on the second diffraction grating are different among the plurality of sets.

18. The measuring device as claimed in claim 11, further comprising:
a collimator lens configured to be positioned between the light source and the introductory part, and collimate light emitted from the light source to illuminate a plurality of introductory parts.

19. A measurement method using a measurement chip, the method comprising:
introducing light into a propagation layer of the measurement chip, the measurement chip comprising the propagation layer configured to allow the light to propagate, an introductory part configured to have a first diffraction grating for introducing the light into the propagation layer, an outgoing part configured to have a second diffraction grating for deriving the light from the propagation layer, and a ligand modification surface configured to be a surface of the propagation layer and capable of modifying a ligand that reacts with an analyte be detected, wherein a period of a plurality of grating patterns formed in at least one of: the first diffraction grating, or the second diffraction grating, is different from each other between two or more regions;
causing the light to totally reflect in the propagation layer which has a ligand layer on a surface of the propagation layer that reacts with the analyte to be detected, and
causing outgoing of the light from the propagation layer.

20. The measuring method as claimed in claim 19. further comprising:
analyzing changes in a pattern of the light outgone from the propagation layer.
